(19) United States
(12) Patent Application Publication
(10) Pub. No.: US 2009/0000629 A1
(43) Pub. Date: Jan. 1, 2009

(54) Elastically Deformable Marker

(76) Inventors: Dirk Hornscheidt, Berlin (DE); Mario Kilka, Schwielochsee OT Goyatz (DE); Frank Kniep, Grossbeeren OT Kleinbeeren (DE); Chris Neumann, Berlin (DE)

Correspondence Address: WARE FRESSOLA VAN DER SLUYS & ADOLPHSON, LLP, BRADFORD GREEN, BUILDING 5, 755 MAIN STREET, P O BOX 224, MONROE, CT, 06468, US

(21) Appl. No.: 11/630,294

(22) PCT Filed: Jun. 23, 2005
(86) PCT No.: PCT/EP05/52935

§ 371 Date: Oct. 29, 2007

(30) Foreign Application Priority Data
Jun. 23, 2004 (DE) 10 2004 030 391.6

(51) Int. Cl. A61B 19/00

(52) U.S. Cl. 128/899; 604/116; 604/523

(57) ABSTRACT

An elastically deformable marker (1) which can be completely implanted by means of a cannula for marking a tissue site, wherein the marker assumes a relaxed condition without an external force acting thereon and has at least one flexurally elastic wire, an elongate central marker portion and a proximal and a distal end portion, wherein a respective one of the two end portions is disposed at one of the two ends of the central marker portion, wherein in the elongate central marker portion at least two portions of the same flexurally elastic wire or a plurality of flexurally elastic wires are twisted together.

### CROSS REFERENCE TO RELATED APPLICATIONS

This application is for entry into the U.S. national phase under §371 for International Application No. PCT/EP2005/052935 having an international filing date of 23 Jun. 2005 and from which priority is claimed under all applicable sections of Title 35 of the United States Code including, but not limited to, Sections 120, 363 and 365(c), and which in turn claims priority under 35 USC §119 to German Patent Application No. 10 2004 030 391.6 filed on 23 Jun. 2004.

### FIELD OF THE INVENTION

The present invention pertains to the field of tissue markers. More particularly, the present invention concerns an elastically deformable marker for marking a tissue site, which can be completely implanted by means of a cannula.

### BACKGROUND OF THE INVENTION

Marking a location in body tissue serves to be able to retrieve the marked tissue site again in subsequent diagnostic investigations and therapeutic interventions by means of imaging processes such as ultrasound, magnetic resonance tomography (MRT) or X-rays. The markers which are involved here serve in particular for marking in postbiotic applications and during neoadjuvant chemotherapy in the region of the breast and in soft part tissue.

Many tumors can be detected and located by means of modern imaging such as X-rays, ultrasound and magnetic resonance tomography (MRT).

In order to be clear whether the situation actually involves a tumor and in order to know what kind of tumor is present tissue is generally taken by means of a biopsy. In the operation of taking tissue, in particular with microcalcifications in the breast the part which is visible in the imaging process used can from time to time also be taken. Without corresponding marking which is visible in medical imaging, the tumor could no longer be localised. Equally a tumor can become invisible for medical imaging during neoadjuvant chemotherapy which from time to time is implemented to shrink a tumor for later surgical removal. By means of the marker, the tumor can in spite of that still be reliably localised.

A whole range of processes and devices for marking and localising given tissue sites are known in the state of the art. For example localisation wire guides as are described in U.S. Pat. No. 5,221,269 for the localisation of diseased locations, specifically in the breast, are well known. The specified device includes a tubular insertion needle and a fitted wire guide which at its distal end is of a coil spring-like configuration in order to anchor it in the proximity of the target tissue. The needle is inserted into the breast and passed by means of an imaging process such as X-ray radiation, ultrasound or magnetic resonance imaging (MRI) to the location at which the distal end is to be disposed. The needle can then be separated from the guide wire which remains in the anchored position in the proximity of the diseased site in order to guide the surgeon to that site during the subsequent operation. Although such a localisation system is highly effective it is nonetheless only intended for temporary use and is removed after termination of the surgical or other intervention.

Other devices are known for marking external regions on the skin of a patient. By way of example U.S. Pat. No. 5,192,270 describes a syringe which delivers a dye in order to provide a visual identification at the surface of that point at which the injection was made.

In a similar fashion U.S. Pat. No. 5,147,307 describes a device having an element for pattern production in order to impress a temporary marking into the skin of the patient in order to specify the position for an injection and the like. It is also known for a small metallic marker, for example a small lead ball measuring 3 mm in diameter to be glued on the skin of a human breast with adhesive strips or in some other fashion in order to identify the position of skin calcifications (see Homer and co-authors, The Geographic Cluster of Microcalcification of the Breast, Surgery, Gynecology & Obstetrics, December 1985).

None of those technical solutions however can be employed for long-term marking and identification of internal tissue abnormalities such as diseased sites or tumors.

Color markings are generally not suitable in the deeper region for non-invasive diagnosis and non-invasive therapy observation.

In addition it is known for markers to be implanted directly in the body of the patient, using invasive processes. For example, in the fitting of a coronary artery bypass (CABG), which naturally takes place on the open heart, the general practice is to fit one or more metallic rings at the location of the implant on the aorta. That makes it possible for the physician later to retrieve the location of the bypass for examination purposes, by localisation of the rings. It is also a current practice for the location of an operation to be marked by means of clamps, vessel clips and the like, for the purposes of future examination.

Accordingly a device for the non-surgical implantation of potentially permanent markers at a diseased site or in abnormal tissue is required in order to define the boundaries of a diseased site before it is removed and/or to establish the location after removal. The markers should be simple to apply and should be easy to detect by means of conventional imaging processes.

A process for the implantation of markers directly in the body of patients using processes of minimally invasive surgery is described in international patent application WO 96/08208. That process provides for introducing a clamping device to the diseased site by means of a tubular cannula. When the clamp is disposed at the diseased site it is fitted by means of an actuating device at the proximal end. That marking device can be used in the long term and can be rendered visible by most imaging processes. Because of its small size however conventional ultrasound imaging systems are not suitable for detecting it in tissue.

Another process for implanting a marker is described in patent application WO 98/06346 bearing the title ‘Apparatus and Method for Marking Tissue’. The marker described for that method comprises a central shank which is biased with a tensile stress in order to cause an end contact bridge which is of a square configuration on the marker to bend the resulting ‘door posts’ arcuately inwardly in order to enclose the tissue. The tensile stress in the shank is increased until it breaks at a predetermined location and leaves behind the marker which is applied at a tissue site. Unfortunately that method requires the marker to be pulled away from the tissue when it is shaped, which limits its penetration and the extent of the tissue detected. Furthermore the marker is passed to the biopsy site through the biopsy cannula which is also used for taking the biopsy sample. It is necessary for the opening of the applicator to be precisely aligned with respect to the biopsy opening on the biopsy cannula in order to fit the marker precisely at the biopsy site.

A surgical clip for permanently connecting mutually opposite tissue portions for anastomosis intervention is described in U.S. Pat. No. 4,733,664. That is effected by an applicator, which is also described, by pulling on a frangible central shank in order to close a pair of arcuate arms which are arranged at a spacing from each other and which extend parallel in a direction from opposite ends of the plastically deformable bridge. The arms are fitted around the oppositely disposed tissue. A predetermined force is applied in order to trigger a stress fracture at a constriction on the shank. Specific angles are specified for the clip shoulders and the applicator. The mutually facing surfaces of the applicator clamping jaws include an angle of between 120° and 180°, preferably 150°. Unfortunately the process for forming that clip suffers from similar disadvantages to the process described in the preceding paragraph. Thus, an applicator is required to pass a biopsy marker to a surgical biopsy site and for placement of the marker at that site. The applicator must be capable of transforming the originally open marker into a closed configuration at the biopsy site. That shaping function however should be simple and reliable to implement. In addition the applicator should easily permit alignment of the opening of the applicator which delivers the marker with respect to the biopsy opening of the biopsy cannula which takes the biopsy sample in order to ensure accurate placement of the marker.

In addition a technical solution is known from EP 1 163 888 ‘Apparatus for the percutaneous marking of a lesion’, wherein suitable forms of markers (circular form, star form, Y-form, tubular form) are introduced by way of a puncturing instrument.

The above-mentioned processes which are known from the state of the art are all highly complicated and expensive and require a high level of material involvement. In addition advantages are incurred by virtue of the fact that marking displacement can occur in placement of the marker as they are not correctly anchored in the tissue to be marked.

### DISCLOSURE OF INVENTION

The aim and object of the invention is to provide a marker which implements marking of a tissue site after insertion of that marker, wherein the marker is intended to permit as accurate placement as possible of the marker and is intended as far as possible to prevent movement of the marker in the tissue after it has been set.

According to the invention that object is attained by a marker which is to be implanted with a cannula and which has a central marker portion which is formed by a twisted wire so that a twist configuration is produced. That twist configuration can be formed by a single wire which is guided back in a loop or by a plurality of wires. Adjoining the two longitudinal ends of the central marker portion are a head part and a base part respectively comprising untwisted portions of the twisted wire or the flexurally elastic wires which are twisted together in the central marker portion. With respect to placement of the marker in the lumen of a cannula, the distal end portion of the marker is identified as the head part while the proximal end portion thereof is identified as the base part.

What is common to all claimed forms of the marker is that they have a central marker portion comprising twisted wire. The twist configuration allows simple manufacture of a marker from one or more wires and imparts to the central marker portion a degree of stability which is increased in relation to the head and base parts. A configuration in which at least two wire portions of the same or a plurality of wires are mutually wound around each other a plurality of times is identified as the twist configuration.

The central marker portion is elongate and in the relaxed condition of the marker is already preferably of a smaller diameter than that of the lumen of a cannula which is usually employed to apply markers. The twist configuration has the further effect that the central marker portion is easier to recognise when using imaging processes.

The twisted wire or the wires which are twisted together in the central marker portion continue outside the central marker portion in untwisted portions and in that way form the head and base parts of the marker. In that case the head and base parts can have elements of two different basic types: thus the head and/or base part can be entirely or partially in the form of a loop or in the form of two or more free or freestanding ends. Combinations comprising one or more loops and free ends are also possible.

The choice of the number of wires to be twisted permits head and/or base parts which adjoin the central marker portion of twisted wire and which have one, two, three, four, five etc free ends. The untwisted wire ends of the wire or wires which is or are twisted in the central marker portion are identified as free or freestanding ends. In the relaxed condition of the marker a free end can be of a straight shape or can be curved in itself, wherein the free end in the relaxed condition does not in any situation lie in a line with an axis through the central marker portion.

A loop is formed from an untwisted portion of a single flexurally elastic wire. In that case that untwisted portion is disposed between two portions of the same wire, which in the central marker portion are twisted together and optionally also to further portions of other wires.

The twisted wire or wires predominantly comprise an elastic or superelastic material. That elastic material can be Nitinol, titanium, high-quality steel or plastic material. A metal alloy which is preferred because of its superelastic properties is Nitinol.

The marker is elastic by virtue of the specified materials so that it can be changed from a relaxed condition in which no external forces act thereon into an elongate shape, with elastic deformation. In that elongate shape, the marker can be inserted into the lumen of a cannula. When it is moved out of the lumen through the opening of the cannula into the body of the patient by means of an ejector, it at least approximately adopts the shape which it possessed previously in the relaxed condition, by virtue of the use of flexurally elastic materials. It is thus possible to use relatively larger markers in spite of small cannula diameters. Insofar as the marker seeks to revert to its original position after insertion into the body tissue to be marked, it does itself exert a force on the body tissue which fixes it in the tissue.

Nitinol is a particularly advantageous material as in MRT it causes lesser artefacts than the high-quality steel wires which are usually employed. It is thus also possible to place a marker using magnetic resonance tomography as the imaging process, and to perform subsequent investigations or post-examinations. The otherwise usual high-quality steel markers in contrast produce too many artefacts so that the placement operation like also all post-examinations are not possible using magnetic resonance tomography.

Two basic forms of the marker according to the invention are particularly preferred. The first is formed in the central marker portion from two mutually twisted wires which at each end respectively terminate in an untwisted portion and thus form a head part and a base part which each have two untwisted free ends; that basic form is also referred to hereinafter as the ‘X-form’. The second preferred basic form is formed by a single flexurally elastic wire, wherein the central marker portion is afforded by twisting two portions of that individual wire. In that case, disposed between the two twisted portions of the wire is an untwisted portion which, as previously described, forms a loop at the base end of the marker. The head end of the marker is then formed by the two free ends of the individual wire. That basic form is also referred to hereinafter as the ‘Y-form’.

The base part and the head part of the marker respectively define surfaces in the following sense:

A loop borders a surface therearound. Two free ends which form at least a portion of a head or base part define between them a surface which is delimited on two sides by the two free ends and which is open on a third side which is delimited by a line extending through the tips of the free ends.

Preferred configurations of the marker according to the invention are distinguished in that at least one surface which is defined or bordered by the head part is not disposed in one plane with a surface which is defined or bordered by the base part. That means that the marker is not flat and assumes a three-dimensional shape. That has the advantage that the marker is particularly well anchored in surrounding tissue and can be better recognised when using imaging processes from different sides as there is no side from which only a flat edge is detected as a line. Particularly preferred marker forms are those in which the planes extending through the surfaces bordered or defined by the head and base parts are approximately or precisely at a right angle to each other.

In order to increase the visibility of the marker when using ultrasound the marker can have a layer or coating with an echogenic substance. The use of porous polytetrafluoroethylene (Teflon) is particularly suitable for that purpose.

Alternatively or additionally the marker can have a medication-bearing layer which contains a medication or a substance used in the known marking processes described in the opening part of this specification for marking body tissue, and which can deliver the medication or substance to the tissue while the marker remains in the tissue.

Preferably the marker according to the invention is of a length of less than 3 cm, better still less than 1 mm or best 5 or fewer millimeters. A typical marker for example is of a length of 6 mm, in the twisted central marker portion it has three complete 360 degree turns, it is of a wire diameter of 0.3 mm and it involves a spacing between the free ends of the head or base parts of 1.5 mm. The surfaces defined by the head and base parts are also preferably turned through 90° relative to each other.

As a marker and the instrument used for inserting it are usually handled as an instrument with the marker already preloaded, the invention also concerns a surgical instrument for the insertion of a marker according to the invention into a body tissue to be marked, which already includes at least one marker according to the invention. Apart from the marker such a surgical instrument is basically designed in known manner and at any event includes a cannula. A preferred surgical instrument is one which has an ejector which can be introduced or is already partially introduced into the lumen of the cannula through an opening provided in the proximal end of the cannula. By the ejector being pushed further into the lumen, the marker which is at the distal cannula opening can be pushed out of the cannula and placed in the body tissue.

A particularly preferred configuration of the surgical instrument provides an ejector which at its distal end has a coupling with which the ejector is releasably connected to the base part, in the form of a loop, of a marker disposed in the lumen. The particular advantage of such a surgical instrument is that the marker can also be moved in the lumen towards the proximal end of the cannula and thus permits retraction of the of the marker and accordingly re-positioning. The coupling can be in the form of a tearable thread, a gripping pincer or a hook. A suitable choice in respect of cannula diameter and the diameter of the loop of the marker in the expanded, that is to say relaxed condition affords a surgical instrument in which the connection between the ejector and the marker can be released after ejection of the marker by a retraction movement of the ejector. That can be implemented by opening a coupling in the form of a pincer-like or hook-like holding device, for example by bending, or also by virtue of destroying the coupling by virtue of the pull on the ejector. It is possible in that way for a marker which has already been placed to be withdrawn into the cannula again (retracted) by a coupling in the form of a suitable gripping mechanism. Such a surgical instrument permits re-positioning or removal of a marker previously placed in the body tissue and therefore represents an invention which is also to be embodied as an independent invention, independently of the specific marker form claimed here.

By virtue of the suitability for re-positioning or removal the embodiment of the marker with a loop at the base part affords particular advantages for that marker in combination with a surgical instrument of the last-mentioned kind.

A particularly advantageous surgical instrument is one which contains a plurality of markers in the lumen of the cannula. Such an instrument has the advantage that a plurality of markers can be set during an operation with a single surgical instrument. So that the markers in the lumen do not hook together at their head and base parts, a spacer comprising a bioresorbable material is respectively arranged between each two adjacent markers. As each spacer preferably at least predominantly comprises a bioresorbable material it can be broken down by the body in a health-compatible fashion with the passage of time after the tissue site has been marked.

A suitable material for the cannula is titanium in regard to use of the surgical instrument in the context of magnetic resonance tomography (MRT).

### BRIEF DESCRIPTION OF THE DRAWINGS

The invention is described hereinafter by way of example by means of an embodiment with reference to the drawings to which moreover reference is expressly directed in regard to the disclosure of all details according to the invention which are not described more fully in the text. In the drawings:

FIG. 1 shows a marker according to the invention,

FIG. 2 shows a surgical instrument according to the invention,

FIG. 3 shows an alternative configuration of a surgical instrument according to the invention,

FIG. 4 shows a variant of the marker according to the invention with half-round pointed wire ends,

FIG. 5 shows a marker according to the invention comprising a single wire in Y-form,

FIG. 6 shows a marker according to the invention in X-form comprising two wires, and

FIG. 7 shows a surgical instrument according to the invention with an ejector which is connected by way of a thread to a base part, in the form of a loop, of the marker of FIG. 5 so that the marker can be retracted.

### DETAILED DESCRIPTION

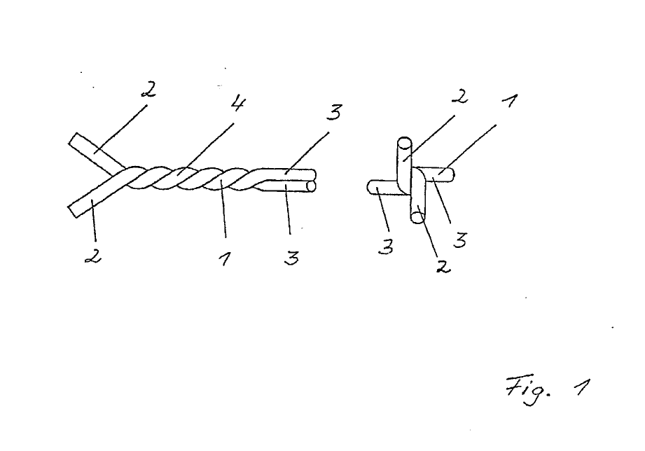

FIG. 1 shows an embodiment according to the invention of a marker in X-form, wherein two Nitinol wires are twisted in such a way that their respective freestanding wire ends 2 and 3 which are referred to hereinafter as the free ends involve three-dimensional orientation. In that case the free ends 2 are oriented turned through about 90° with respect to the free ends 3. That orientation for the free ends 2 and 3 can be seen in FIG. 1 as a plan view on to the marker 1. The specific X-form of the marker 1 enjoys the advantage that the marker is particularly well visible from any perspective for an imaging process as it does not appear as a simple line when seen from any perspective. In addition, it is fixed to prevent movements of the marker in any direction in the tissue after insertion, by virtue of the free ends disposed at both ends of the marker.

By virtue of its structure the marker can be used in a cannula of an outside diameter of for example 0.95 mm or 1.2 mm.

The free ends 2 and 3 can be of a blunt, preferably rounded configuration. That provides for better compatibility in sensitive tissues. Alternatively grinding the free wire ends so they are sharp or shaping them to a point can facilitate or first permit placement in insensitive hard tissue.

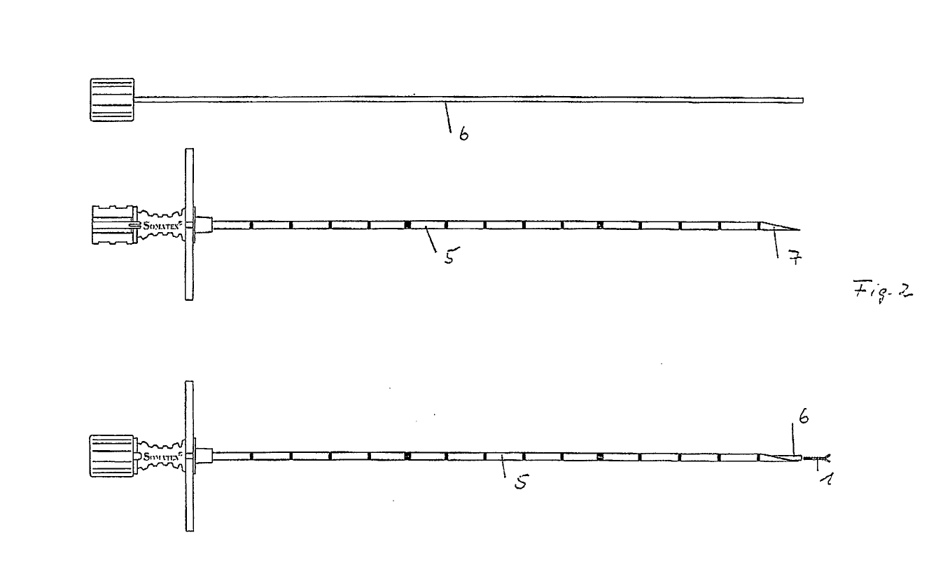

FIG. 2 shows a surgical instrument which is known per se apart from the included marker according to the invention and which can be used for setting the marker 1. It has a cannula 5 which is provided with a ground surface 7. Provided on the cannula 5 are markings for determining the puncture depth when inserting the cannula into body tissue. The marker 1 is placed in the cannula 5 in the lumen thereof near a distal cannula opening. An internal bar portion for placement in the cannula 5 of a length which is of such a magnitude that it terminates immediately before the marker 1 is placed in the lumen of the cannula 5 in front of the marker from the proximal point of view. The internal bar portion provides that, upon puncturing with the cannula 5, the pierced body tissue does not penetrate into the cannula 5 and urge the marker 1 back into the cannula 5.

After placement of the cannula 5 in the tissue to be marked the internal bar portion is removed and an ejector 6 is inserted. The ejector 6 is of such a length that it extends as far as the end of the cannula 5 or projects slightly beyond same. By insertion of the ejector 6 the marker is pushed out of the cannula 5 and positioned in the body tissue to be marked directly at the point of location of the distal end of the cannula 5 and the cannula opening.

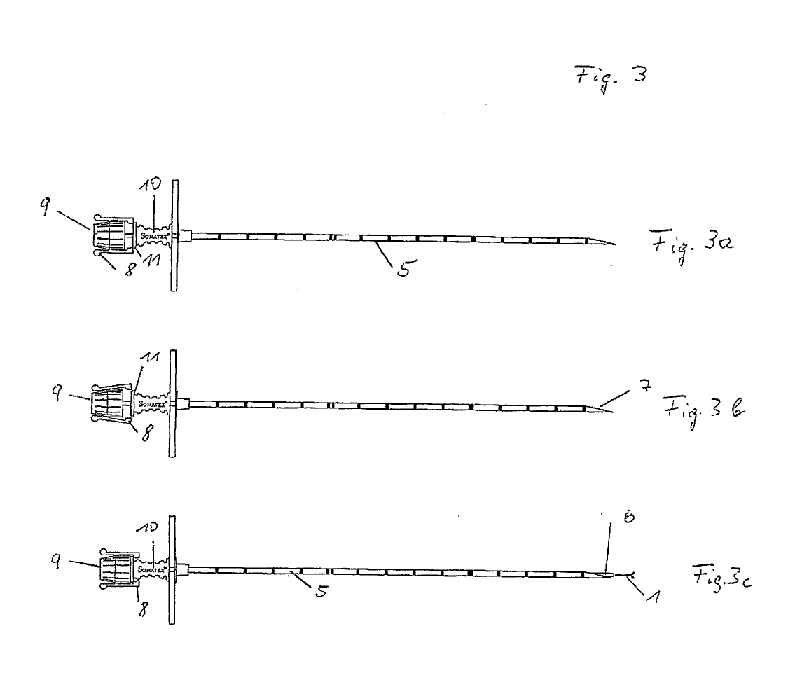

FIG. 3 shows an alternative embodiment of a surgical instrument which has a cannula gripping portion 10. A bar portion with a bar gripping portion 9 is introduced into that cannula gripping portion 10 in such a way that the bar portion is placed by way of a spacing latch 8 in front of an arresting edge 11 of the cannula gripping portion 10. In that way the bar portion is suitable for simultaneously performing the function of the ejector and of the internal bar portion referred to in the description relating to FIG. 2. Upon positioning the cannula 5 in body tissue, the insertion depth of the bar portion into the lumen of the cannula 5, which is predetermined by the spacing latch 8, prevents the marker 1 from being prematurely placed in the body tissue; at the same time body tissue which penetrates into the lumen of the cannula 5 cannot urge the marker 1 deeper into the lumen, by virtue of the presence of the bar portion. That is diagrammatically shown in FIG. 3.

It can be seen from FIG. 3b how the spacer latch 8 can be opened by pressing the ends thereof in order to be able to move the bar portion beyond the arresting edge 11 further into the lumen of the cannula 5 to act as an ejector and to place the marker 1 in the tissue. FIG. 3c shows the end condition after placement of the marker 1 has been effected, wherein the spacing latch 8 latches over the arresting edge of the cannula gripping portion 11.

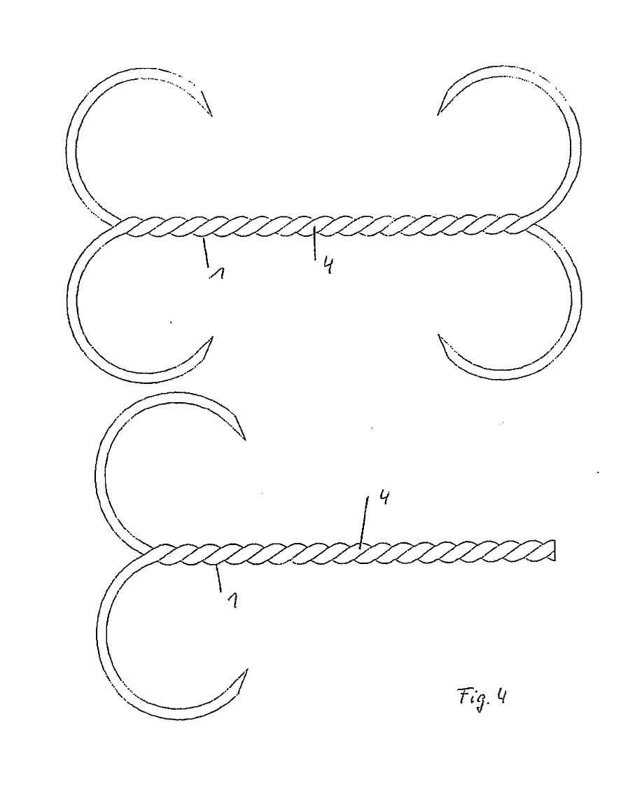

FIG. 4 shows two different embodiments of the marker 1 according to the invention which each have a twist configuration 4 of two wires. Both embodiments have freestanding ends which are curved in a round or half-round shape in the relaxed condition, wherein the first form, the X-form, has two free ends at each end of the marker 1 while the second form, the Y-form, has two free ends only at one end. There is also the possibility of an individual marker 1 simultaneously having freestanding ends of a straight shape and of a pre-bent round shape. The forms with straight freestanding wire ends are particularly advantageous for soft tissue.

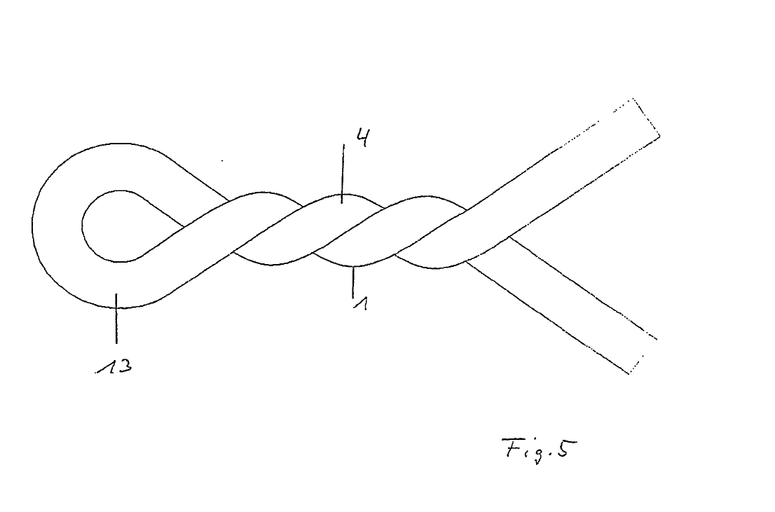

FIG. 5 shows a further embodiment of the marker 1 according to the invention in which a single wire is twisted in such a way that a loop 13 is formed at one longitudinal end of the marker and two freestanding ends are formed at the other longitudinal end of the marker. That marker form corresponds to a variant of the Y-form of the marker 1, which can be re-positioned because of the loop.

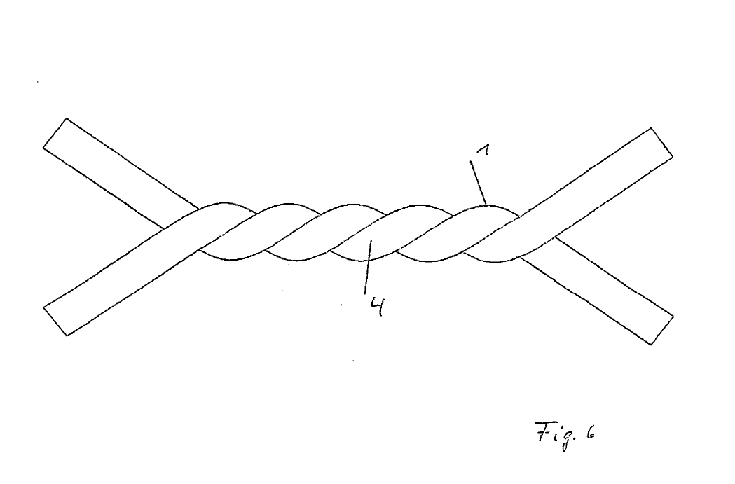

FIG. 6 shows a marker 1 which has a twist configuration formed by two wires, wherein the free ends are of a straight shape. The X-form can be seen in the plan view illustrated. Besides the illustrated form, further forms are also possible, thus for example an embodiment in which three wires are twisted together and which thus has a total of six free ends which are arranged in the same plane or in a three-dimensional orientation. The twist configuration 4 formed from wires of an elastic material such as Nitinol, titanium, high-quality steel or plastic material provides that mechanical forces which occur when the marker 1 is expelled are better absorbed and fracture of the free ends of the marker 1 is prevented.

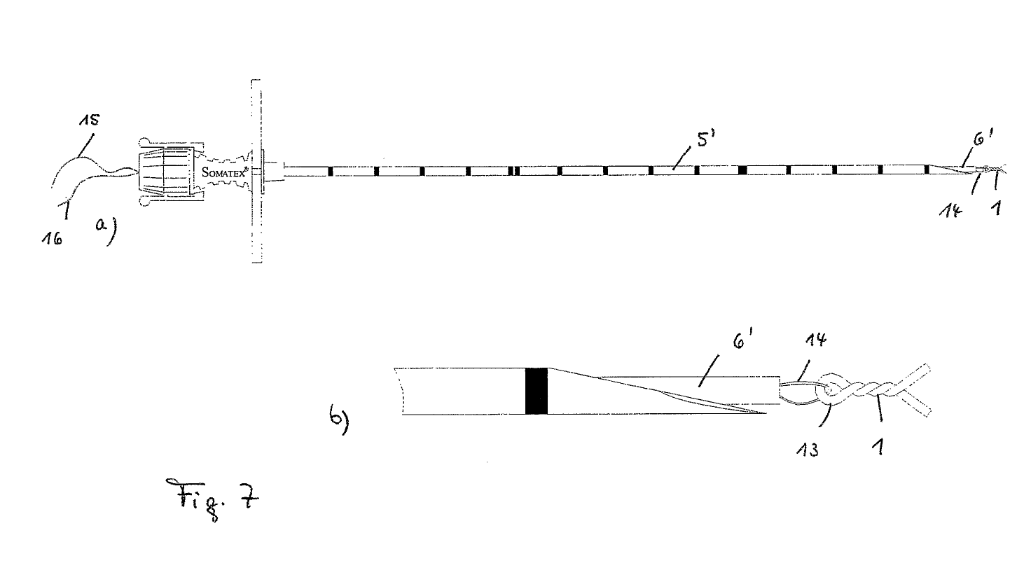

FIG. 7 shows a surgical instrument which is substantially the same as the surgical instrument shown in FIG. 2. In that respect FIG. 7a shows the complete surgical instrument as a side view while FIG. 7b is an enlarged view of the distal end of the surgical instrument.

The surgical instrument shown in FIG. 7 differs from the surgical instrument of FIG. 2 in that the ejector 6′ is of a tubular configuration and has a lumen through which a thread 14 is passed from the proximal end of the ejector 6′ to the distal end thereof and back again to the proximal end of the ejector 6′. A loop of the thread 14 projects distally out of the ejector 6′ (see FIG. 7b) and engages into the loop 13 of the marker 1. The marker 1 moreover corresponds to the marker shown in FIG. 5, in its Y-form. The marker 1 can be pulled back into the cannula 5′ of the surgical instrument in FIG. 7 again by means of the thread 14 in order to be able to re-position the marker 1. When finally the marker 1 is to be released one of the free ends 15 or 16 of the thread 14, that are accessible at the proximal end of the surgical instrument, can be released and the thread 14 with the respective other free end 15 or 16 can be pulled out of the lumen of the ejector 6′.

What is claimed is:

1. An elastically deformable marker (1) which can be completely implanted by means of a cannula for marking a tissue site, wherein the marker has a flexurally elastic wire, an elongate central marker portion and a proximal and a distal end portion, wherein a respective one of the two end portions is disposed at one of the two ends of the central marker portion,
characterised in that
in the elongate central marker portion at least two portions of the same flexurally elastic wire or a plurality of flexurally elastic wires are twisted together.

2. A marker as set forth in claim 1 characterised in that at least one end portion has at least one loop formed by an untwisted portion of the flexurally elastic wire or one of the flexurally elastic wires.

3. A marker as set forth in one of claims 1 and 2 characterised in that at least of the end portions has at least two untwisted free ends of the flexurally elastic wire or a plurality of flexurally elastic wires.

4. A marker as set forth in claim 3 characterised in that
the central marker portion has two flexurally elastic wires which are twisted together, and
that each of the end portions of the marker has an untwisted free end of each of the two flexurally elastic wires.

5. A marker as set forth in claims 2 and 3 characterised in that
the central marker portion is formed by two portions which are twisted together of a single flexurally elastic wire,
that the proximal end portion of the marker is in the form of a loop, wherein the loop is formed from an untwisted portion of the single flexurally elastic wire, which untwisted portion connects the two portions which are twisted together, and
the distal end portion of the marker is formed by two untwisted free ends of the single flexurally elastic wire.

6. A marker as set forth in one of claims 3 through 5 characterised in that at least one of the untwisted free ends is of a pointed configuration.

7. A marker as set forth in one of claims 3 through 6 characterised in that at least one of the untwisted free ends is curved in a relaxed condition of the marker.

8. A marker as set forth in one of claims 1 through 7 characterised in that the flexurally elastic wire or the flexurally elastic wires predominantly comprise plastic material.

9. A marker as set forth in one of claims 1 through 7 characterised in that the flexurally elastic wire or wires predominantly comprise a metal.

10. A marker as set forth in claim 9 characterised in that the metal is a superelastic metal such as Nitinol.

11. A marker as set forth in claim 9 characterised in that the metal is selectively high-quality steel or titanium.

12. A marker as set forth in one of claims 1 through 11 characterised in that at least one surface which is bordered or defined by the distal end portion in the relaxed condition of the marker is not disposed in one plane with a surface which is bordered or defined by the proximal end portion in the relaxed condition.

13. A marker as set forth in claim 12 and as set forth in one of claims 4 and 5 characterised in that the surface defined by the two untwisted free ends of the distal end portion in a relaxed condition of the marker is turned with respect to the surface bordered or defined by the two untwisted free ends or the loop of the proximal end portion in the relaxed condition, through an angle of rotation of between 80 and 100°, about the main axis of the elongate central marker portion.

14. A marker as set forth in claim 13 characterised in that the angle of rotation is 90°.

15. A marker as set forth in one of claims 1 through 14 characterised in that the marker has an echogenic coating.

16. A marker as set forth in claim 15 characterised in that the echogenic coating contains porous polytetrafluoroethylene.

17. A marker as set forth in one of claims 1 through 16 characterised in that a medication-bearing layer is applied on at least a part of the surface of the marker.

18. A marker as set forth in one of claims 1 through 17 characterised in that the marker is of a length of less than 3 cm.

19. A marker as set forth in claim 18 characterised in that the marker is of a length of less than 1 cm.

20. A marker as set forth in claim 19 characterised in that the marker is of a length of 6 mm or less.

21. A surgical instrument comprising
a cannula for insertion into body tissue, which encloses a lumen which is open towards the distal end of the cannula through a distal cannula opening and which is radially delimited by an inside wall of the cannula and which extends along the longitudinal axis of the cannula, and
at least one marker which is disposed in the lumen and which is displaceable along the longitudinal axis of the cannula, and having a proximal and a distal end portion, wherein the distal end portion of the at least one marker is disposed closer to the distal end of the cannula than the proximal end portion of the marker,
characterised in that the at least one marker is a marker as set forth in one of claims 1 through 20.

22. A surgical instrument as set forth in claim 21 characterised in that
the lumen of the cannula is opened to the proximal end of the cannula in a proximal cannula opening, and
the surgical instrument has an ejector which can be introduced or is entirely or partially introduced into the lumen through the proximal cannula opening.

23. A surgical instrument as set forth in claim 22 characterised in that
precisely one marker which is a marker as set forth in claim 5 is disposed in the lumen of the cannula, and
at its distal end the ejector has a coupling which is releasably connected or connectable to the proximal end portion of the one marker, which proximal end portion is in the form of a loop.

24. A surgical instrument as set forth in one of claims 21 and 22 characterised in that
disposed in the lumen of the cannula are a plurality of markers which are arranged in succession along the longitudinal axis of the cannula, and
a respective spacer of a bioresorbable material is disposed between each two adjacent markers.

25. A surgical instrument as set forth in one of claims 21 through 24 characterised in that the cannula comprises titanium.
